(19) United States
(12) Patent Application Publication
(10) Pub. No.: US 2025/0169983 A1
(43) Pub. Date: May 29, 2025

(54) CANALOPLASTY VISCOELASTIC DELIVERY

(71) Applicant: Alcon Inc., Fribourg (CH)

(72) Inventors: Wayne Noda, Mission Viejo, CA (US); Daniel Hyman, Foothill Ranch, CA (US); David Kimball, Irvine, CA (US); Jestwin Edwin Lee, IV, Grandview, TX (US)

(21) Appl. No.: 18/956,532

(22) Filed: Nov. 22, 2024

Related U.S. Application Data
(60) Provisional application No. 63/603,380, filed on Nov. 28, 2023.

(51) Int. Cl. A61F 9/00

(52) U.S. Cl. CPC A61F 9/0008

(57) ABSTRACT

An apparatus for controlling or metering a viscoelastic material for eye surgery. Such an apparatus may generally comprise a housing having a cylinder; a plunger having at least a portion operable to move within the cylinder; a reservoir; an inlet check; and an outlet check valve fluidly coupled to the reservoir. The inlet check valve may be configured to allow the viscoelastic material into the reservoir at a priming pressure. An actuator can be coupled to the plunger and configured to move the plunger in a first direction to increase the pressure in the reservoir to a delivery pressure that is sufficient to close the inlet check valve and open the outlet check valve. A return spring can be configured to move the plunger in a second direction to decrease the pressure in the reservoir.

### PRIORITY CLAIM

This application claims the benefit of priority of U.S. Provisional Patent Application Ser. No. 63/603,380 titled “CANALOPLASTY VISCOELASTIC DELIVERY”, filed on Nov. 28, 2023, whose inventor(s) is/are Wayne Noda, Daniel Hyman, David Kimball and Jestwin Edwin Lee, IV, which is hereby incorporated by reference in its entirety as though fully and completely set forth herein.

### TECHNICAL FIELD

The invention set forth in the appended claims relates generally to delivery of viscoelastic fluid for eye surgery, including, without limitation, delivery of viscoelastic fluid for canaloplasty to treat glaucoma.

### BACKGROUND

The human eye can suffer a number of maladies causing mild deterioration to complete loss of vision. For example, glaucoma is a condition of increased pressure in an eye, and researchers have theorized that prolonged exposure to high intraocular pressure can damage the optic nerve that transmits sensory information from the eye to the brain. This damage to the optic nerve can result in loss of peripheral vision. As glaucoma progresses, more and more of the visual field is lost until vision is completely lost. For this reason, eye care professionals routinely screen patients for glaucoma by measuring intraocular pressure.

It is commonly believed that pressure inside the eye can begin to rise if natural drainage systems in the eye stop functioning properly. Thus, treatments for glaucoma often focus on relieving the pressure. Canaloplasty is one surgical technique that can promote outflow of aqueous via the natural drainage systems through the trabecular meshwork and into Schlemm's canal.

While the benefits of canaloplasty for treating glaucoma are known, improvements to delivery systems, components, and processes continue to improve outcomes and benefit patients.

### BRIEF SUMMARY

New and useful systems, apparatuses, and methods for delivery of viscoelastic fluid for eye surgery are set forth in the appended claims. Illustrative embodiments are also provided to enable a person skilled in the art to make and use the claimed subject matter.

For example, some embodiments comprise an apparatus for controlling or metering a viscoelastic material for eye surgery. Such an apparatus may generally comprise a housing having a cylinder; a plunger having at least a portion operable to move within the cylinder; a reservoir; an inlet check valve; and an outlet check valve fluidly coupled to the reservoir. The inlet check valve may be configured to allow the viscoelastic material into the reservoir at a priming pressure. An actuator can be coupled to the plunger and configured to move the plunger in a first direction to increase the pressure in the reservoir to a delivery pressure that is sufficient to close the inlet check valve and open the outlet check valve. A return spring can be configured to move the plunger in a second direction to decrease the pressure in the reservoir.

In some embodiments, the apparatus may additionally comprise a source of viscoelastic material; a cannula configured to be inserted into an eye; a first conduit fluidly coupled to the inlet check valve and the source of viscoelastic material; and a second conduit fluidly coupled to the outlet check valve. The second conduit can be configured to be extended through the cannula into the eye. The source of viscoelastic material can be pressurized at the priming pressure in some embodiments. Additionally, or alternatively, a conduit controller may be coupled to the second conduit and configured to extend the second conduit through the cannula.

Additionally, or alternatively, some embodiments may comprise a lower chassis configured to support the actuator; an upper chassis; and an end cap enclosing a portion of the lower chassis and the upper chassis.

In other aspects, some embodiments may comprise a housing having a first chassis, a second chassis, a cylinder, and a reservoir. The first chassis may comprise an inlet flow path, and the second chassis may comprise an outlet flow path. The reservoir may be coupled to the cylinder, the inlet flow path, and the outlet flow path. A plunger may have at least a portion disposed within the cylinder, and an actuator can be configured to move the plunger within the cylinder. An outlet check valve can be coupled to the outlet flow path, and an inlet check valve can be coupled to the inlet flow path. A seal can be disposed between the first chassis and the second chassis, and an end cap can enclose a portion of the first chassis and the second chassis. A snap hook can be configured to hold the first chassis and the second chassis together without adhesives.

In yet other aspects, some embodiments may comprise a housing having a reservoir; a piston comprising a piston seat and a piston channel fluidly coupled to the reservoir; and a piston seal slidingly disposed in the piston seat. An actuator can be configured to move the piston from a first piston position to a second piston position within the housing. The piston seal can be configured to move from a first seal position to a second seal position within the piston seat if the actuator moves the piston from the first piston position to the second piston position. The piston channel is open if the piston seal is in the first seal position, and the piston channel is closed if the piston seal is in the second seal position. In some embodiments, a source of viscoelastic material can be pressurized at a priming pressure, wherein the priming pressure is sufficient to allow the viscoelastic material to move through the channel into the reservoir if the piston seal is in the first seal position. Additionally, moving the piston from the first piston position to the second piston position can increase the pressure in the reservoir to a delivery pressure sufficient to displace the viscoelastic material from the reservoir through an outlet. In more particular embodiments, a first conduit can be fluidly coupled to the source of viscoelastic material, and a second conduit can be fluidly coupled to the reservoir. A cannula can be configured to be inserted into an eye, and the second conduit configured to be extended through the cannula into the eye. Moving the piston from the first piston position to the second piston position can displace the viscoelastic material from the reservoir through the second conduit. In some embodiments, a conduit controller can be coupled to the second conduit and configured to extend the second conduit through the cannula.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is a schematic view of an interior of a human eye.

FIG. 2 is a schematic representation of an apparatus being used by a physician to treat the eye of a patient.

FIG. 3 is a schematic view of another example of the apparatus of FIG. 2.

FIG. 4 is schematic view of a distal end of a cannula that may be associated with some embodiments of the apparatus of FIG. 3.

FIG. 5 is a schematic view of another example of the apparatus.

FIG. 6 is a perspective view of an example of a flow controller that may be associated with some embodiments of the apparatus.

FIG. 7 is a schematic side view of the flow controller of FIG. 6.

FIG. 8 is a schematic top view of the flow controller of FIG. 6.

FIG. 9 is a perspective view of another example of the flow controller.

FIG. 10 is another view of the flow controller of FIG. 9.

FIG. 11 is a section view of the flow controller of FIG. 9.

FIG. 12 is a schematic view of another example of the flow controller.

FIG. 13 is a perspective view of another example of the flow controller.

FIGS. 14A-14C are schematic diagrams of examples of an actuator that may be associated with the apparatus.

FIG. 15 is a side view of another example of the flow controller.

FIG. 16 is a schematic section view of another example of the flow controller.

FIG. 17 is a side view of a piston that may be associated with the flow controller of FIG. 16.

FIG. 18 is a schematic view of the flow controller of FIG. 16 in a second configuration.

FIG. 19 is a schematic section view of another example of the flow controller.

FIG. 20 is a schematic section view of the flow controller of FIG. 19 in a second configuration.

FIG. 21A and FIG. 21B are side views of an example of a visco module that may be associated with some examples of the apparatus.

FIG. 22 is a transparent view of the visco module of FIG. 21B.

FIG. 23 is a section view of the visco module of FIG. 22.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient receiving treatment. However, as should be recognized by those skilled in the art, this frame of reference is merely a descriptive expedient rather than a strict prescription.

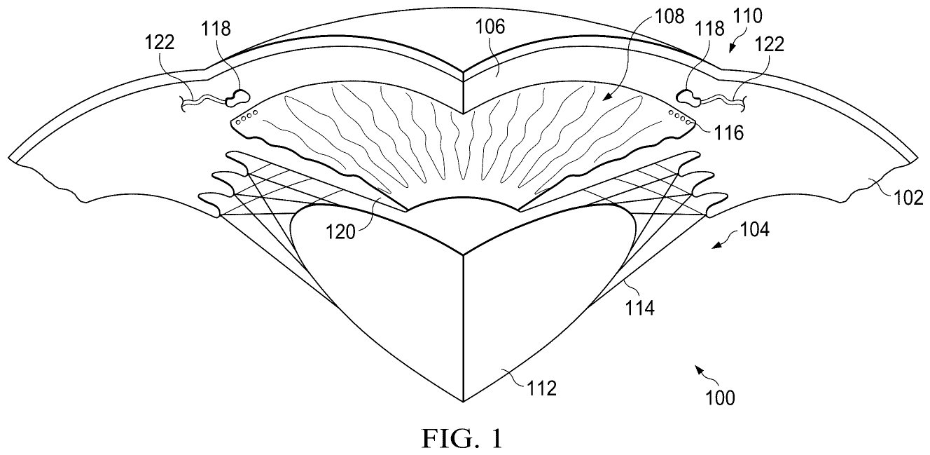

FIG. 1 is a schematic view of an interior of a human eye 100. Eye 100 can be conceptualized as a fluid filled ball having two chambers. Sclera 102 of eye 100 surrounds a posterior chamber 104 filled with a viscous fluid known as vitreous humor. Cornea 106 of eye 100 encloses an anterior chamber 108 that is filled with a fluid know as aqueous humor. The cornea 106 meets sclera 102 at a limbus 110 of eye 100. A lens 112 of eye 100 is located between anterior chamber 108 and posterior chamber 104. Lens 112 is held in place by a number of ciliary zonules 114. Whenever a person views an object, that object is viewed through the cornea, the aqueous humor, and the lens of the eye. To be transparent, the cornea and the lens can include no blood vessels. Accordingly, no blood flows through the cornea and the lens to provide nutrition to these tissues and to remove wastes from these tissues. Instead, these functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the eye as new aqueous humor is secreted by the epithelial cells of the ciliary body. This excess aqueous humor enters the blood stream and is carried away by venous blood leaving the eye.

In a healthy eye, aqueous humor flows out of the anterior chamber 108 through the trabecular meshwork 116 and into Schlemm's canal 118, located at the outer edge of the iris 120. Aqueous humor exits Schlemm's canal 118 by flowing through a number of outlets 122. After leaving Schlemm's canal 118, aqueous humor is absorbed into the venous blood stream.

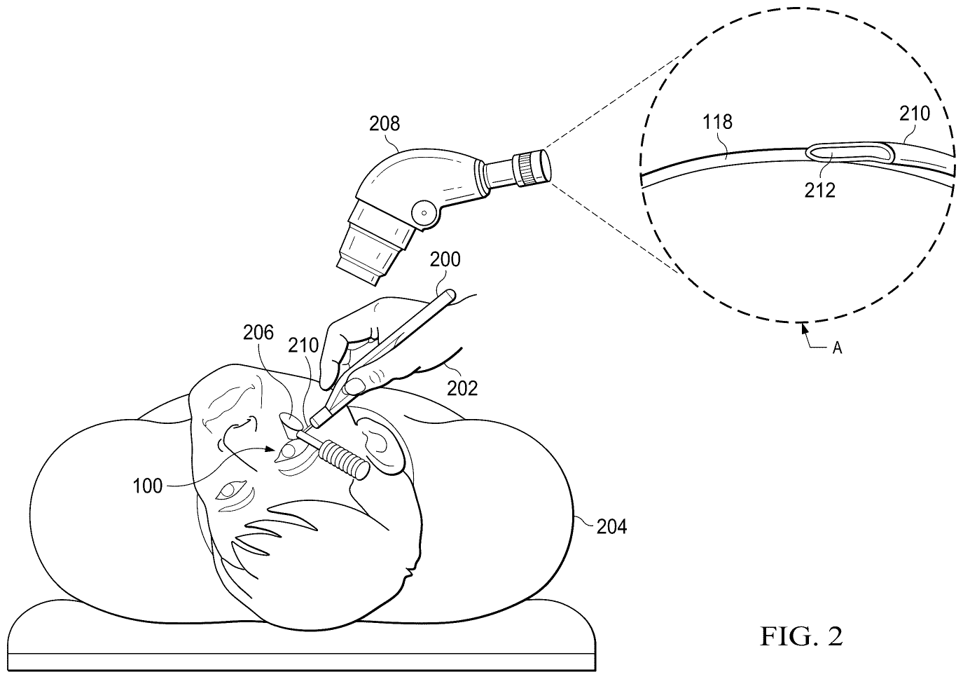

FIG. 2 is a schematic representation of an apparatus 200 being used by a physician 202 to treat the eye 100 of a patient 204. In the example procedure of FIG. 2, the apparatus 200 is held in the right hand of the physician 202. The left hand (not shown) may be used to hold the handle of a gonio lens 206. Alternatively, some physicians may prefer holding the apparatus 200 in the left hand and the gonio lens 206 in the right hand.

During the example procedure of FIG. 2, the physician 202 may view the interior of the anterior chamber 108 using a gonio lens 206 and a microscope 208. Detail A of FIG. 2 is a stylized representation of an image that may be viewed by the physician 202 through the microscope 208. For example, a distal portion of a cannula 210 of the apparatus 200 is visible in Detail A of FIG. 2, as well as the location of Schlemm's canal 118, which is lying under various tissues (e.g., the trabecular meshwork 116) that surround the anterior chamber 108. A distal opening 212 of cannula 210 can be positioned near Schlemm's canal 118 of the eye 100.

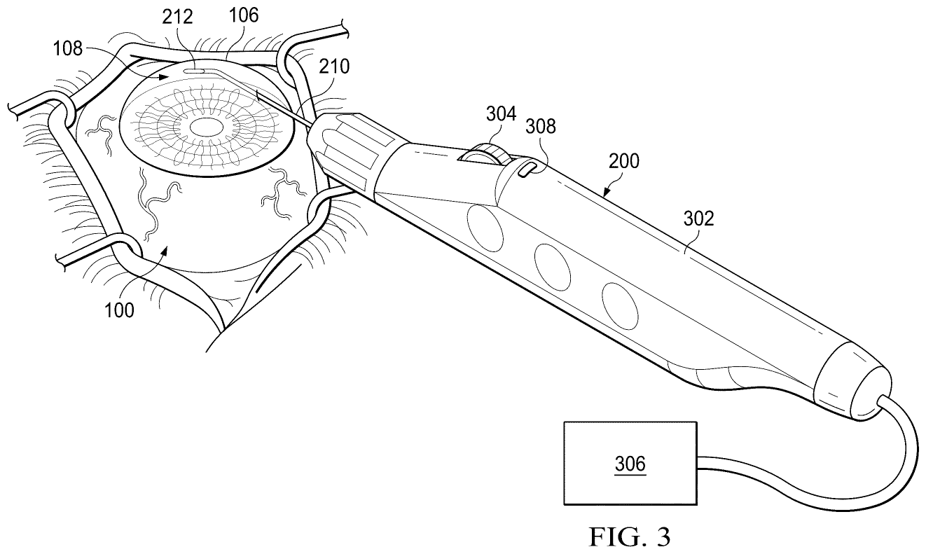

FIG. 3 is a schematic view of another example of the apparatus 200, illustrating additional details that may be associated with some embodiments. For example, the cannula 210 of FIG. 3 extends from a handle 302 and through the cornea 106 of the eye 100. A distal portion of the cannula 210 may be disposed inside the anterior chamber 108 defined by the cornea 106 of the eye 100. In the example of FIG. 3, the cannula 210 may be configured so that the distal opening 212 of cannula 210 can be placed in fluid communication with Schlemm's canal 118 (not visible in FIG. 3).

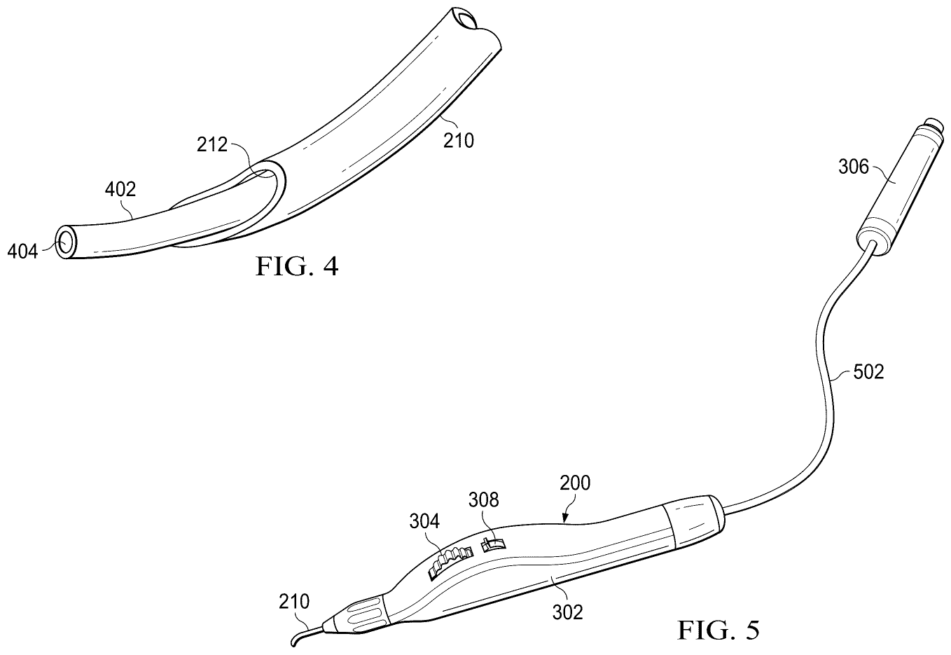

In a canaloplasty procedure, a viscoelastic material can be administered through the cannula 210 into Schlemm's canal, which can dilate Schlemm's canal to promote outflow of aqueous via the natural outflow pathways through the trabecular meshwork. For example, in some embodiments, a conduit, such as a microcatheter (see FIG. 4) can be disposed within the cannula 210. In some embodiments, such a conduit can be configured to be advanced out of the cannula 210 into Schlemm's canal, and viscoelastic material can be delivered into Schlemm's canal from the conduit. The apparatus 200 of FIG. 3 includes a conduit controller, such as a wheel 304, which can be configured to advance and/or retract a conduit along the cannula 210. For example, the wheel 304 can be rotated in a first direction (e.g., towards the cannula 210) to advance the conduit towards a distal tip of the cannula 210 and partially out of the cannula 210 into Schlemm's canal when the distal end of the cannula 210 is in fluid communication with Schlemm's canal. Furthermore, moving the wheel the in a second direction (e.g., away from the cannula 210) can retract the conduit within the cannula 210 and to withdraw it within and from Schlemm's canal.

In some examples, the viscoelastic material, also commonly known as an ophthalmic viscosurgical device (OVD), may be delivered into Schlemm's canal by advancing the conduit through the distal opening 212 of the cannula 210 while the distal opening 212 is in fluid communication with Schlemm's canal. Viscoelastic material can then be administered from the conduit into Schlemm's canal. The viscoelastic material can be delivered into Schlemm's canal of the eye before or after delivering an ocular implant into the eye of the patient.

In some embodiments, the apparatus 200 may include or can be fluidly coupled to a source of viscoelastic material, such as a visco module 306. The visco module 306 can store viscoelastic material and can be configured to deliver viscoelastic material into the conduit within the cannula 210. In one implementation, the apparatus 200 can include an actuator 308, such as a lever, button, or similar device, which can be configured to release viscoelastic material from the visco module 306 into the conduit.

The wheel 304 can enable the conduit to be moved within Schlemm's canal without administering any viscoelastic material from the conduit. Likewise, the actuator 308 can allow viscoelastic material to be administered from the conduit into Schlemm's canal without moving the conduit.

FIG. 4 is schematic view of a distal end of the cannula 210, illustrating additional details that may be associated with some embodiments. In the example of FIG. 4, a conduit 402 is shown partially extending from the distal opening 212 of the cannula 210. As described above, the conduit 402 can be slidably disposed within the cannula 210 and can be advanced partially beyond the distal opening 212 of the cannula 210 (such as with the wheel 304).

A “conduit,” in this context, broadly includes a tube, pipe, hose, or other structure with one or more lumina or open pathways adapted to convey a fluid or viscoelastic material between two ends. Typically, a tube is an elongated, cylindrical structure with some flexibility, but the geometry and rigidity may vary. Moreover, some conduits may be molded into or otherwise integrally combined with other components.

The conduit 402 may be made of a polyethylene, polyurethane, polyamide, or other suitable material. For example, Grilamid® or VESTAMID® polyamide or Pebax® elastomer may be suitable for some embodiments. In some embodiments, the conduit 402 can have a size and a cross-sectional shape that matches the size and cross-sectional shape of the interior of the cannula 210. For example, an outside diameter of 0.008 inch and inside diameter of 0.006 inch may be suitable for some embodiments. Suitable cross-sections may be circular or oval, for example. More generally, the conduit 402 can be any shape so long as it is able to be slidably disposed within the cannula 210. The conduit 402 can have a sufficient length to enable it to extend beyond the distal tip of the cannula 210 half-way around Schlemm's canal. For example, a length of about of 16-22 millimeters may be particularly suitable for some embodiments. The conduit 402 can also include a distal opening 404, which can be configured to deliver a viscoelastic material. The conduit 402 generally includes a lumen that fluidly communicates with a source of viscoelastic material (such as visco module 306) to facilitate administration of viscoelastic material. While the illustrative example of FIG. 4 includes only the distal opening 404, other embodiments of the conduit 402 may include additional openings, such as openings along a side of the conduit 402.

FIG. 5 is a schematic view of another example of the apparatus 200, illustrating additional details that may be associated with some embodiments. As shown in the example of FIG. 5, a conduit 502 may couple the apparatus 200 to the visco module 306 in some embodiments. The conduit 502 may be similar or analogous to the conduit 402.

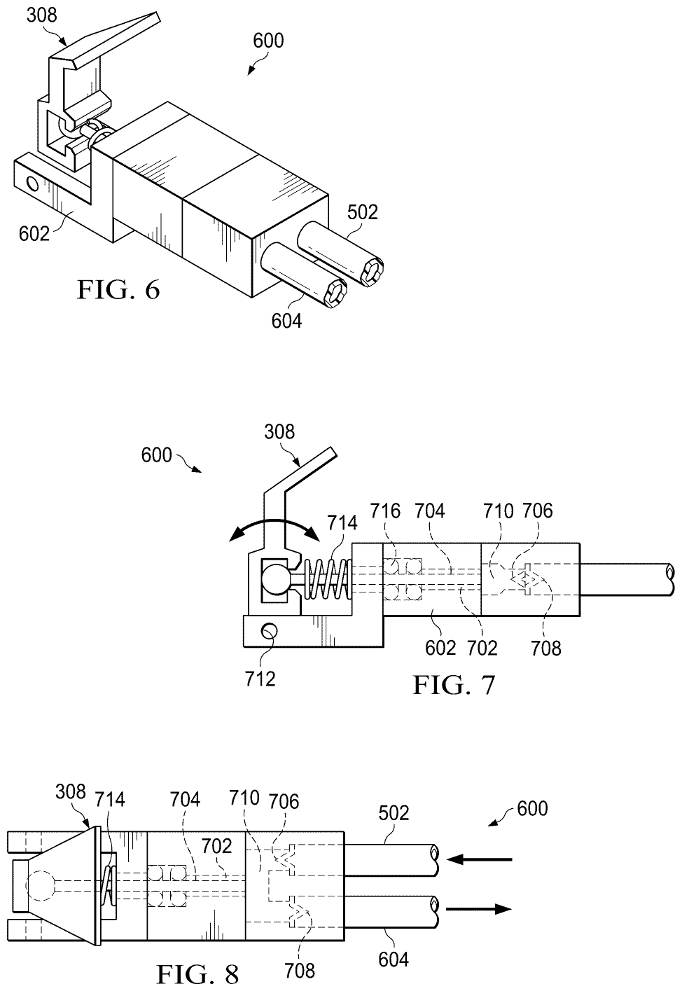

FIG. 6 is a perspective view of an example of a flow controller 600, which may be associated with some embodiments of the apparatus 200. For example, the flow controller 600 of FIG. 6 may include or be coupled to the actuator 308 for controlled metering of viscoelastic material in some embodiments. As shown in FIG. 6, the actuator 308 may be operatively coupled to a housing 602. In some embodiments, the housing 602 may be enclosed within the handle 302, and the actuator 308 may protrude through an opening in the handle 302, as shown in the example of FIG. 5. The housing 602 may be coupled to the conduit 502 and to a conduit 604.

FIG. 7 is a schematic side view of the flow controller 600 of FIG. 6, illustrating additional details that may be associated with some embodiments. For example, the housing 602 of FIG. 7 comprises a cylinder 702. The flow controller 600 of FIG. 7 also comprises a plunger 704, an inlet check valve 706, an outlet check valve 708, and a reservoir 710 fluidly coupled to the cylinder 702, the inlet check valve 706, and the outlet check valve 708. In some embodiments, the inlet check valve 706 and the outlet check valve 708 may be duckbill valves, as illustrated in FIG. 7. The plunger 704 of FIG. 7 has at least a portion operable to move within the cylinder 702. A pivot pin 712 may couple the actuator 308 to the housing 602, and the actuator 308 may be coupled to the plunger 704 and a return spring 714. Seals 716 between the plunger 704 and the cylinder 702 can provide a barrier to prevent transfer of fluid or viscoelastic material from the reservoir 710 through the cylinder 702, while allowing the plunger 704 to move. In some examples, the seals 716 may be ring seals made of silicone.

FIG. 8 is a schematic top view of the flow controller 600 of FIG. 6, illustrating additional details that may be associated with some embodiments. For example, the conduit 502 can be coupled to a source of fluid or viscoelastic material, such as the visco module 306 (not shown in FIG. 8), which can be pressurized.

For example, the visco module 306 may maintain viscoelastic material at a priming pressure. In a priming configuration, the priming pressure is sufficient to open the inlet check valve 706, allowing the viscoelastic material into the reservoir 710. The actuator 308 can be configured to move the plunger 704 in a first direction to increase the pressure in the reservoir 710 from a first pressure to a second pressure, which can close the inlet check valve 706 and open the outlet check valve 708 to displace the fluid or viscoelastic material from the reservoir 710 through the conduit 604. For example, the actuator 308 of FIG. 8 can move the plunger 704 toward the outlet check valve 708 to increase the pressure in the reservoir 710 from a priming pressure to a delivery pressure. The return spring 714 can return the actuator 308 and the plunger 704 to the initial positions, which can reduce the pressure in the reservoir 710. The diameter and travel distance of the plunger 704 determines the volume of material that can be delivered by each movement of the actuator 308. In other examples, the source of fluid or viscoelastic material may not be pressurized, or sufficiently pressurized, and the actuator 308 can move the plunger 704 in another direction to decrease the pressure in the reservoir 710, which can cause fluid or viscoelastic material to flow into the reservoir 710 through the inlet check valve 706.

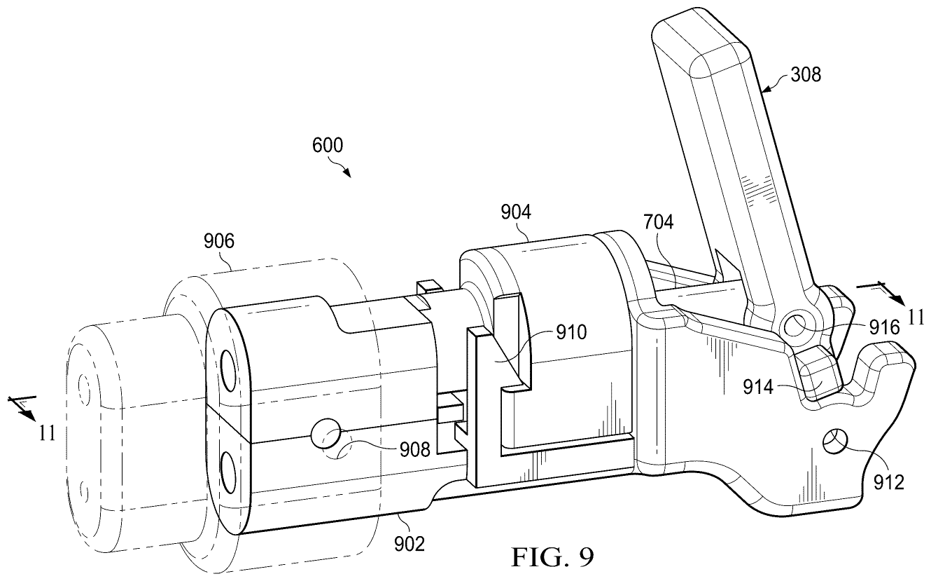

FIG. 9 is a perspective view of another example of the flow controller 600, illustrating additional details that may be associated with some embodiments. For example, the flow controller 600 may comprise a first chassis and a second chassis, such as a lower chassis 902 and an upper chassis 904 illustrated in FIG. 9. The lower chassis 902 can provide support for the actuator 308. An end cap 906 captures both the lower chassis 902 and the upper chassis 904. For example, the end cap 906 of FIG. 9 encloses a portion of the end of each of the lower chassis 902 and the upper chassis 904, which can keep them aligned and adjacent to one another. The end cap 906 may have cross holes 908 through which a dowel or pin (not shown) can be inserted to lock all the chassis components together. A snap hook 910 can provide a locking feature to hold the lower chassis 902 and the upper chassis 904 together. Thus, in the example of FIG. 9, the flow controller 600 may be assembled without adhesives.

A pin (not shown) though hole 912 can couple the actuator 308 to the lower chassis 902. A travel stop block 914 can limit the movement of the actuator 308 between stops. The flow controller 600 may also have a return spring, similar or analogous to the return spring 714 of FIG. 7, which can return the actuator 308 to the original position. A pin (not shown) through a hole 916 can couple the plunger 704 to the actuator 308.

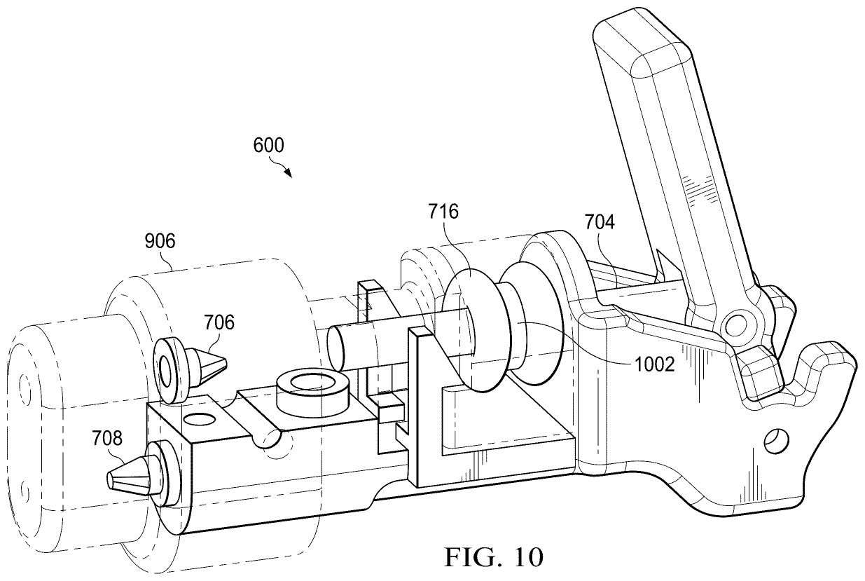

FIG. 10 is another view of the flow controller 600 with the upper chassis 904 removed to illustrate additional details that may be associated with some embodiments. For example, as shown in FIG. 10, the upper chassis 904 may contain the inlet check valve 706, and the lower chassis 902 may contain the outlet check valve 708. In some embodiments, the inlet check valve 706 and the outlet check valve 708 can be captured and sealed by compression of the end cap 906. The plunger 704 can be sealed by the seals 716 with spacer 1002 in between.

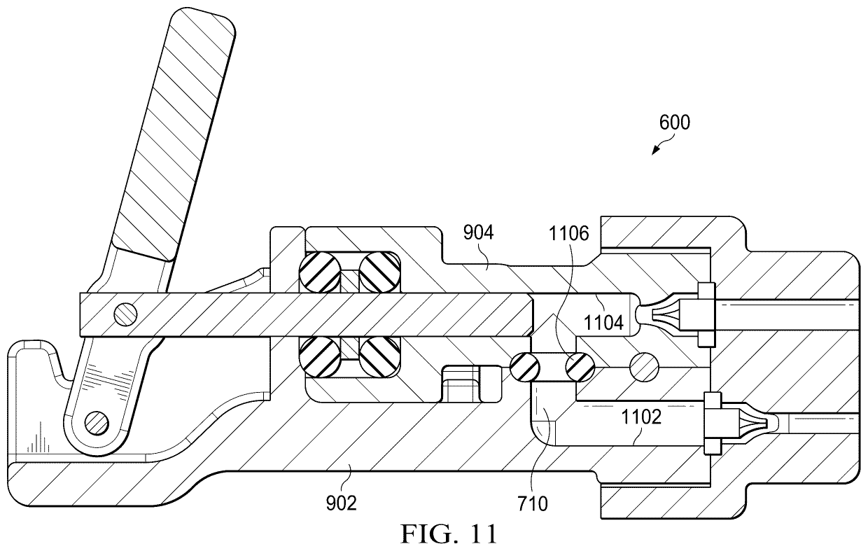

FIG. 11 is a section view of the flow controller 600 of FIG. 9 taken along line 11-11, illustrating additional details that may be associated with some embodiment. As shown in FIG. 11, the lower chassis 902 may contain an outlet flow path 1102, and the upper chassis 904 may contain an inlet flow path 1104. The reservoir 710 may be disposed between, and fluidly couple, the inlet flow path 1104 and the outlet flow path 1102. A ring seal 1106 can provide a seal between upper and lower chassis components.

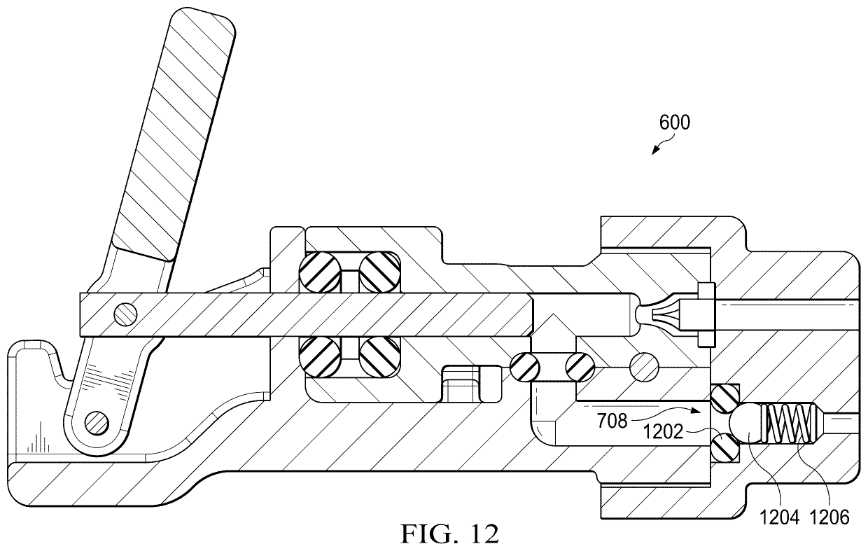

FIG. 12 is a schematic view of another example of the flow controller 600, illustrating additional details that may be associated with some embodiments. For example, the outlet check valve 708 of FIG. 12 comprises or consists essentially of a ball check valve assembly instead of a duckbill valve. The outlet check valve 708 of FIG. 12 generally includes of a ring seal 1202, a ball 1204, and a compression spring 1206. The outlet check valve 708 of FIG. 12 may be advantageous for achieving a higher cracking pressure to prevent unintended flow from a pressurized source of viscoelastic material.

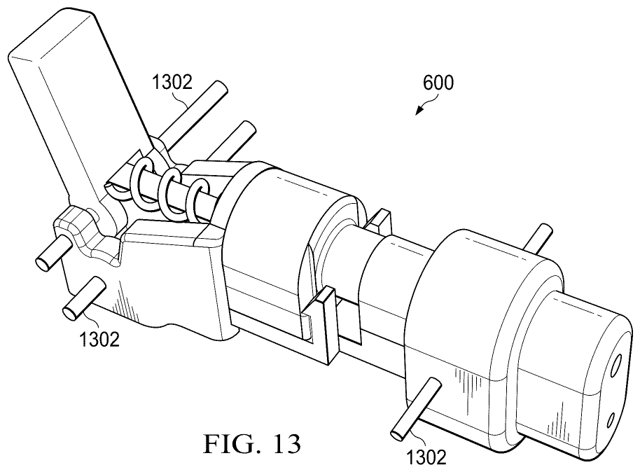

FIG. 13 is a perspective view of another example of the flow controller 600, illustrating additional details that may be associated with some embodiments. For example, the flow controller 600 of FIG. 13 illustrates pins 1302, which can be used to assemble the flow controller 600 without adhesives.

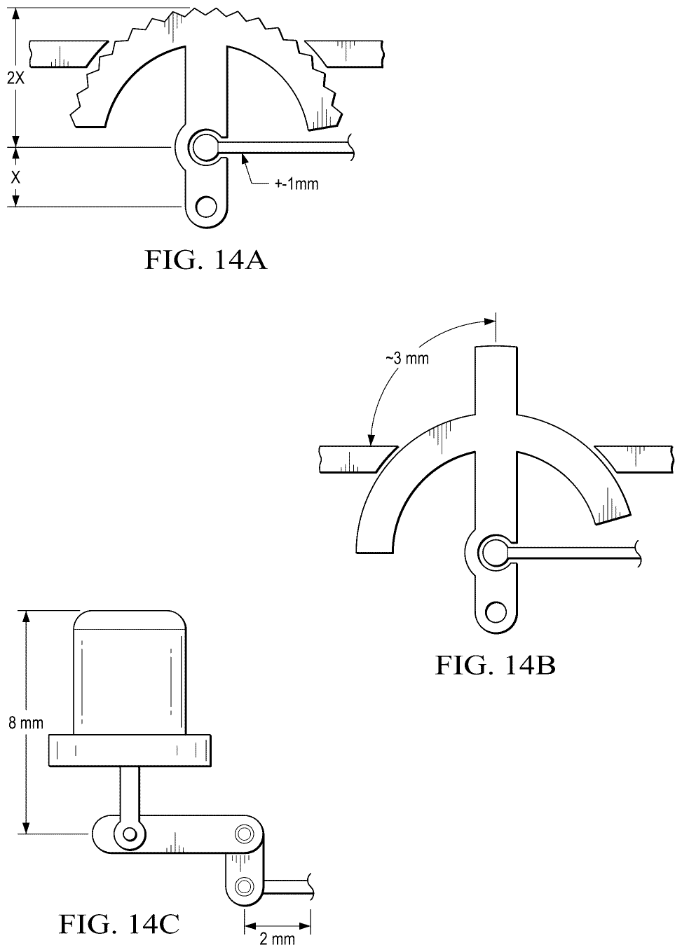

FIGS. 14A-14C are schematic diagrams of other examples of the actuator 308, illustrating additional details that may be associated with some embodiments. For example, the actuator 308 of FIG. 14A is a rolling wheel actuator; FIG. 14B is a lever actuator; and FIG. 14C is a button actuator. Each of these examples can be configured to provide a suitable mechanical advantage for actuating the plunger 704. In some embodiments, for example, a mechanical advantage of 4:1 may be suitable.

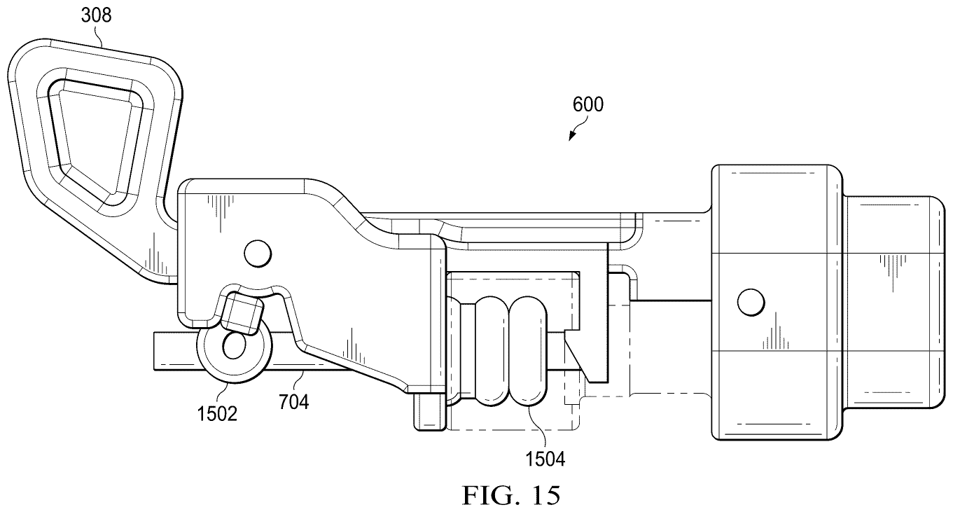

FIG. 15 is a side view of another example of the flow controller 600, illustrating additional details that may be associated with some embodiments. For example, the actuator 308 of FIG. 15 is a button actuator. The button may be coupled to the plunger 704 at an attachment point 1502 to form a bell crank structure with a suitable mechanical advantage. The example of FIG. 15 also illustrates another seal 1504, which may be made from a harder and lubricious material, such as TEFLON, to serve as a wiper seal. In some examples, the seal 1504 can substantially reduce or prevent excess viscoelastic material from reaching the seals 716.

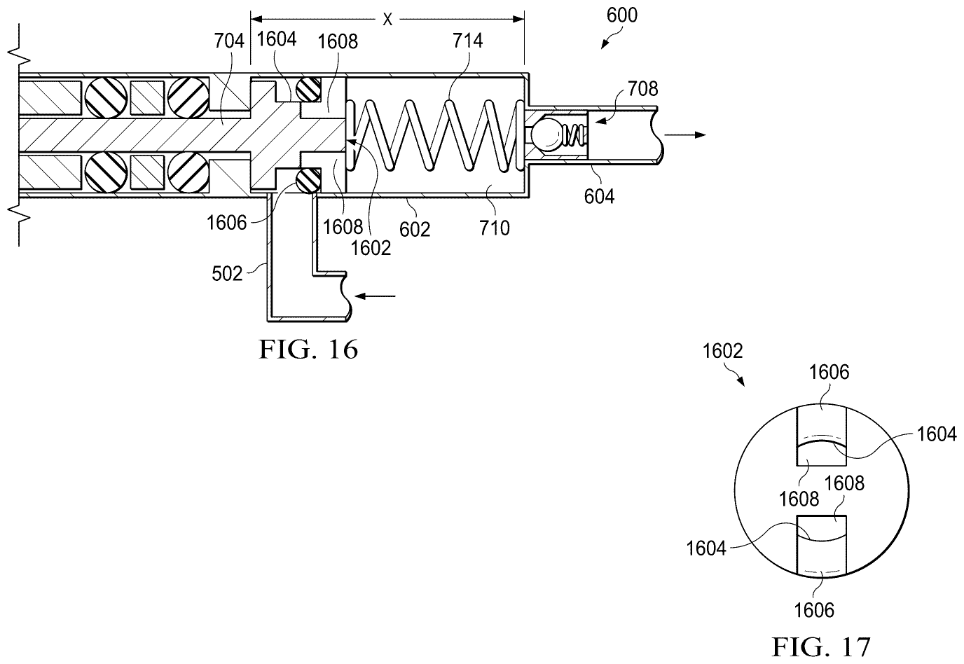

FIG. 16 is a schematic section view of another example of the flow controller 600, illustrating additional details that may be associated with some embodiments in a first configuration. For example, the flow controller 600 may have an inlet, which can be coupled through the conduit 502 to a source of fluid or viscoelastic material, such as the visco module 306 (not shown in FIG. 16). In the example of FIG. 16, the flow controller 600 comprises a piston 1602, which may be coupled to the plunger 704. The return spring 714 can be disposed between the piston 1602 and the housing 602.

The piston 1602 of FIG. 16 comprises a seat 1604. In the example of FIG. 16, the seat 1604 comprises an annular recess, notch, groove, or similar channel around the piston 1602. A piston seal 1606 may be disposed in the seat 1604. A distal end of the piston 1602 of FIG. 16 has a channel 1608, which can fluidly couple the conduit 502 to the reservoir 710. In the configuration of FIG. 16, the channel 1608 is open and the piston seal 1606 can allow transfer of fluid or viscoelastic material from the conduit 502 to the reservoir 710. For example, the visco module 306 can be sufficiently pressurized so that the fluid or viscoelastic material flows around edges of the piston 1602 to the seat 1604, and then between the piston seal 1606 and the piston 1602 into the channel 1608 and to the reservoir 710.

FIG. 17 is a side view of the piston 1602 of FIG. 16, illustrating additional details that may be associated with some embodiments. For example, as shown in FIG. 17, the channel 1608 may form or comprise a recess, notch, or groove in the distal end of the piston 1602, below or inboard of the seat 1604.

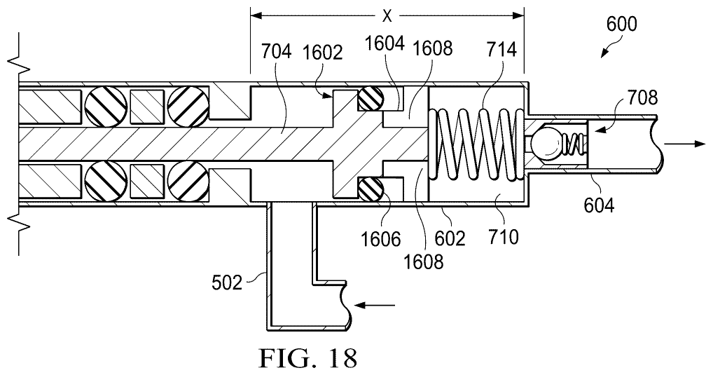

FIG. 18 is a schematic view of the flow controller 600 of FIG. 16 in a second configuration. For example, an actuator, such as the actuator 308 (not shown in FIG. 17), can be configured to move the plunger 704, and thus the piston 1602, from a first position illustrated in FIG. 16 to a second position illustrated in FIG. 18. Additionally, the piston seal 1606 may be configured to slide within the seat 1604 from the first position of FIG. 16, in which the channel 1608 is open, to a second position, as illustrated in FIG. 18, in which the channel 1608 is closed. For example, as the piston 1602 moves from the first position to the second position, friction between the piston seal 1606 and the housing 602 can cause the piston seal 1606 to move relative to the seat 1604. In the closed position of FIG. 18, the piston seal 1606 can provide a barrier to prevent transfer of fluid or viscoelastic material from the conduit 502 to the reservoir 710.

Moving the piston 1602 from the first position to the second position can increase the pressure in the reservoir 710 from a first pressure to a second pressure, which can displace the fluid or viscoelastic material from the reservoir 710 through an outlet to the conduit 604. In the example of FIG. 18, moving the piston 1602 to the second position compresses the return spring 714. If the actuator 308 is released or reversed, the return spring 714 can return the piston 1602 to the initial position, which can reduce the pressure in the reservoir 710 to the first pressure. The diameter and travel distance of the piston 1602 determines the volume of material that can be delivered by each actuation of the actuator 308.

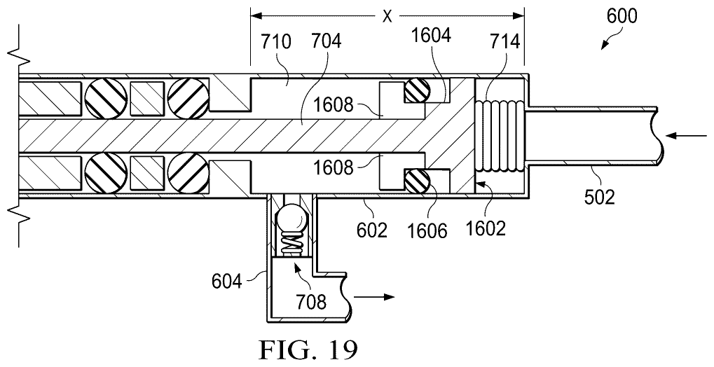

FIG. 19 is a schematic section view of another example of the flow controller 600, illustrating additional details that may be associated with some embodiments. Analogous to the example of FIG. 16, the flow controller 600 of FIG. 19 is shown in a first configuration that allows fluid or viscoelastic material to flow around the piston 1602 to the reservoir 710. For example, the visco module 306 (not shown in FIG. 19) can be sufficiently pressurized so that the fluid or viscoelastic material flows from the conduit 502 around edges of the piston 1602 to the seat 1604, and then between the piston seal 1606 and the channel 1608 to the reservoir 710.

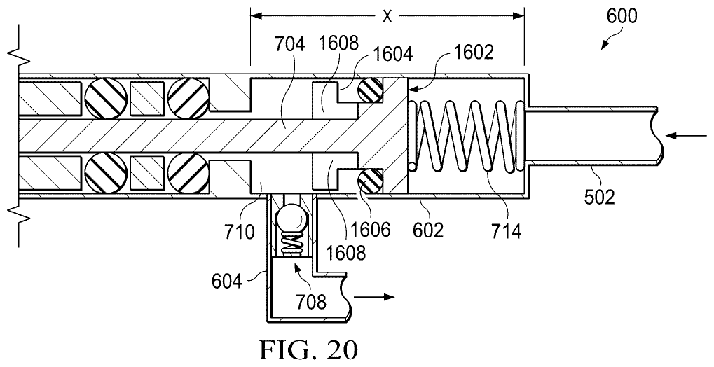

FIG. 20 is a schematic section view of the flow controller 600 of FIG. 19 in a second configuration, analogous to the example of FIG. 18. For example, an actuator, such as the actuator 308 (not shown in FIG. 20), can be configured to move the plunger 704, and thus the piston 1602, from a first position illustrated in FIG. 19 to a second position illustrated in FIG. 20. Additionally, the piston seal 1606 may be configured to slide within the seat 1604 from the first position of FIG. 19 to a second position, as illustrated in FIG. 20. For example, as the piston 1602 moves from the first position to the second position, friction between the piston seal 1606 and the housing 602 can cause the piston seal 1606 to move relative to the seat 1604. In the second position of FIG. 20, the piston seal 1606 can provide a barrier to prevent transfer of fluid or viscoelastic material from the conduit 502 to the reservoir 710.

Moving the piston 1602 from the first position to the second position can increase the pressure in the reservoir 710 from a first pressure to a second pressure, which can displace the fluid or viscoelastic material from the reservoir 710 through the conduit 604. More particularly, in the example of FIG. 20, the return spring 714 is compressed in when the piston 1602 is in the first position. If the actuator 308 is released or reversed, the return spring 714 can move the piston 1602 to the second position, which can increase the pressure in the reservoir 710 to the second pressure. The actuator 308 can be activated or reversed to return the piston 1602 to the first position. The diameter and travel distance of the piston 1602 determines the volume of material that can be delivered by each actuation of the actuator 308.

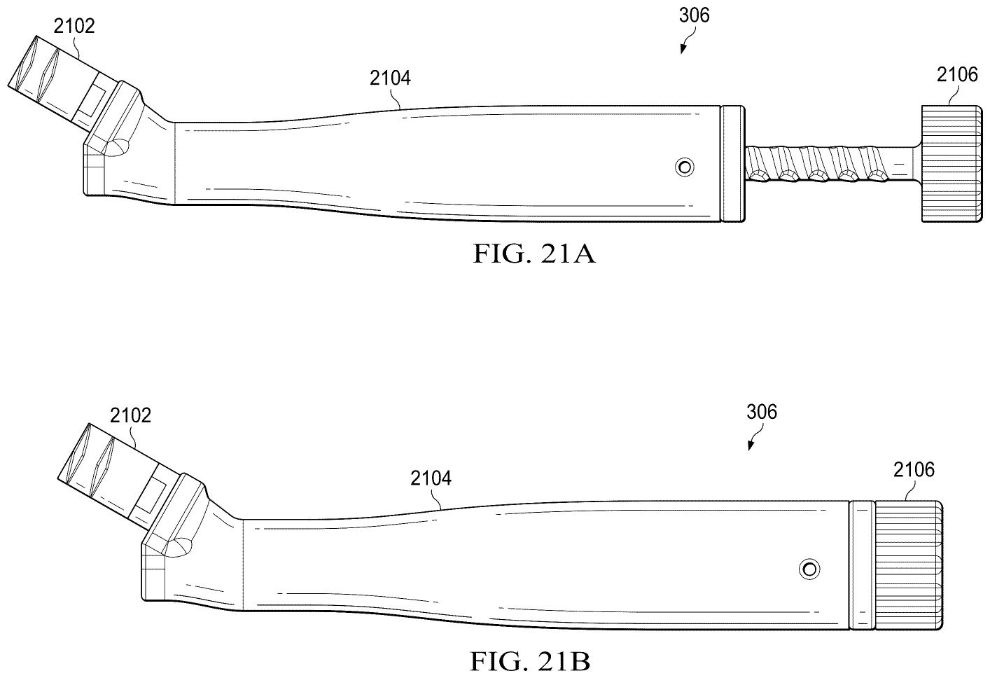

FIG. 21A and FIG. 21B are side views of an example of the visco module 306, illustrating additional details that may be associated with some embodiments. In the example of FIG. 21A and FIG. 21B, a connector port 2102 may be attached to a body 2104. The connector port 2102 can provide an inlet for filling the visco module 306 with visco elastic material. Once filled, a pressure screw 2106 can be tightened, as shown in FIG. 21B.

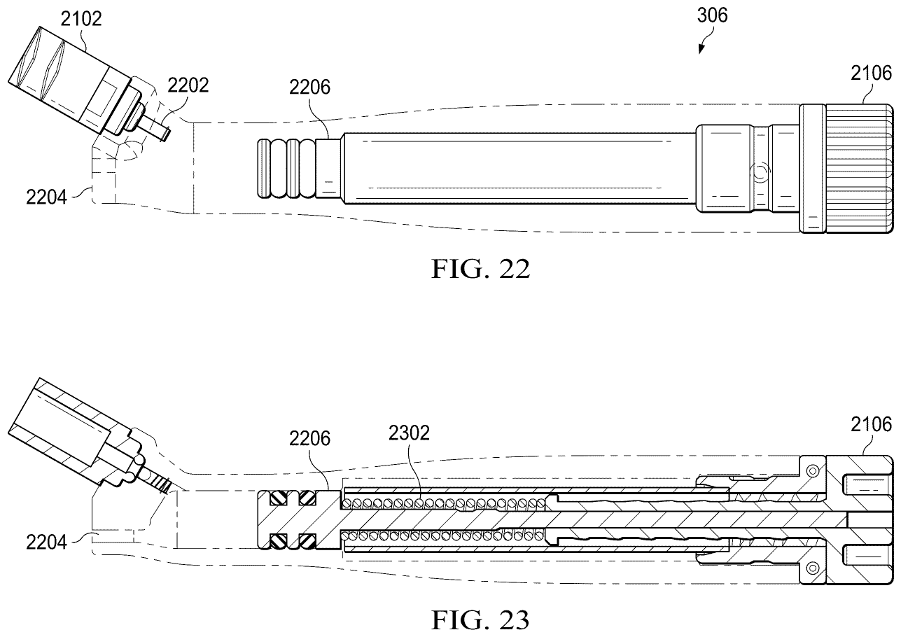

FIG. 22 is a transparent view of the visco module 306 of FIG. 21B, illustrating additional details that may be associated with some embodiments. For example, a check valve 2202 can prevent leakage out of the connector port 2102 in some embodiments. In the example of FIG. 22, the check valve 2202 is a ball check valve. An outlet 2204 can be connected to a delivery apparatus, such as the apparatus 200. The visco module 306 of FIG. 22 also includes a piston 2206, which can be advanced to force viscoelastic material through the outlet 2204.

FIG. 23 is a section view of the visco module 306 of FIG. 22, illustrating additional details that may be associated with some embodiments. For example, the visco module 306 may include a compression spring 2302, which can be compressed to place a force on the piston 2206 if the pressure screw 2106 is tightened.

The systems, apparatuses, and methods described herein may provide significant advantages. For example, some embodiments can provide a consistent and fixed volume delivery of viscoelastic material for various surgical procedures, including canaloplasty.

While shown in a few illustrative embodiments, a person having ordinary skill in the art will recognize that the systems, apparatuses, and methods described herein are susceptible to various changes and modifications that fall within the scope of the appended claims.

Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations, the flow controller 600 and/or the visco module 306 may be combined or sold separately.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for metering a viscoelastic material for eye surgery, the apparatus comprising:
a housing comprising a cylinder;
a plunger having at least a portion operable to move within the cylinder;
a reservoir;
an inlet check valve configured to allow the viscoelastic material into the reservoir at a priming pressure;
an outlet check valve fluidly coupled to the reservoir; and
an actuator coupled to the plunger, the actuator configured to move the plunger in a first direction to increase pressure in the reservoir to a delivery pressure that is sufficient to close the inlet check valve and open the outlet check valve.

2. The apparatus of claim 1, further comprising a return spring configured to move the plunger in a second direction to decrease pressure in the reservoir.

3. The apparatus of claim 1, further comprising:
a source of viscoelastic material;
a cannula configured to be inserted into an eye;
a first conduit fluidly coupled to the inlet check valve and the source of viscoelastic material; and
a second conduit fluidly coupled to the outlet check valve, the second conduit configured to be extended through the cannula into the eye.

4. The apparatus of claim 3, wherein the source of viscoelastic material is pressurized at the priming pressure.

5. The apparatus of claim 3, further comprising a conduit controller coupled to the second conduit and configured to extend the second conduit through the cannula.

6. The apparatus of claim 1, wherein the priming pressure is less than the delivery pressure.

7. The apparatus of claim 1, further comprising:
a lower chassis configured to support the actuator;
an upper chassis; and
an end cap enclosing a portion of the lower chassis and the upper chassis.

8. An apparatus for metering a viscoelastic material for eye surgery, the apparatus comprising:
a housing comprising:
a first chassis having an inlet flow path,
a second chassis having an outlet flow path,
a cylinder, and
a reservoir coupled to the cylinder, the inlet flow path, and the outlet flow path;
a plunger having at least a portion within the cylinder;
an actuator configured to move the plunger within the cylinder;
an outlet check valve coupled to the outlet flow path;
an inlet check valve coupled to the inlet flow path;
a seal between the first chassis and the second chassis;
an end cap enclosing a portion of the first chassis and the second chassis; and
a snap hook configured to hold the first chassis and the second chassis together.

9. The apparatus of claim 8, further comprising:
a source of viscoelastic material;
a cannula configured to be inserted into an eye;
a first conduit fluidly coupled to the inlet check valve and the source of viscoelastic material; and
a second conduit fluidly coupled to the outlet check valve, the second conduit configured to be extended through the cannula into the eye.

10. The apparatus of claim 9, wherein:
the inlet check valve is configured to allow the viscoelastic material into the reservoir at a priming pressure; and
the source of viscoelastic material is pressurized at the priming pressure.

11. The apparatus of claim 10, wherein the actuator is configured to move the plunger in a first direction to increase pressure in the reservoir to a delivery pressure that is sufficient to close the inlet check valve and open the outlet check valve.

12. The apparatus of claim 11, wherein the delivery pressure is greater than the priming pressure.

13. The apparatus of claim 11, further comprising a return spring configured to move the plunger in a second direction to decrease pressure in the reservoir.

14. An apparatus for metering a viscoelastic material for eye surgery, the apparatus comprising:
a housing having a reservoir;
a piston comprising a piston seat and a piston channel fluidly coupled to the reservoir;
a piston seal slidingly disposed in the piston seat; and
an actuator configured to move the piston from a first piston position to a second piston position within the housing;
wherein the piston seal is configured to move from a first seal position to a second seal position within the piston seat if the actuator moves the piston from the first piston position to the second piston position, the piston channel is open if the piston seal is in the first seal position, and the piston channel is closed if the piston seal is in the second seal position.

15. The apparatus of claim 14, further comprising a source of viscoelastic material that is pressurized at a priming pressure; wherein the priming pressure is sufficient to allow the viscoelastic material to move through the piston channel into the reservoir if the piston seal is in the first seal position.

16. The apparatus of claim 15, wherein moving the piston from the first piston position to the second piston position increases pressure in the reservoir to a delivery pressure sufficient to displace the viscoelastic material from the reservoir through an outlet.

17. The apparatus of claim 15, further comprising:
a cannula configured to be inserted into an eye;
a first conduit fluidly coupled to the source of viscoelastic material; and
a second conduit fluidly coupled to the reservoir, the second conduit configured to be extended through the cannula into the eye;
wherein moving the piston from the first piston position to the second piston position displaces the viscoelastic material from the reservoir through the second conduit.

18. The apparatus of claim 17, further comprising a conduit controller coupled to the second conduit and configured to extend the second conduit through the cannula.

19. The apparatus of claim 16, further comprising a return spring configured to move the piston to the second piston position to decrease pressure in the reservoir.

20. The apparatus of claim 14, wherein the piston seat comprises an annular recess in the piston.

21. (canceled)
